(19) United States
(12) Patent Application Publication
(10) Pub. No.: US 2006/0217467 A1
(43) Pub. Date: Sep. 28, 2006

(54) Salt-like reaction products of hals derivatives and carboxylic acids for the stabilisation of polymeric materials

(76) Inventor: Peter Staniek, Binzen (DE)

Correspondence Address: CLARIANT CORPORATION;INTELLECTUAL PROPERTY DEPARTMENT, 4000 MONROE ROAD, CHARLOTTE, NC, 28205, US

(21) Appl. No.: 10/546,501

(22) PCT Filed: Feb. 19, 2004
(86) PCT No.: PCT/IB04/00509

§ 371 Date: Aug. 19, 2005

(30) Foreign Application Priority Data
Feb. 21, 2003 (EP) 03003939.0

(51) Int. Cl. C08K 5/34; C07D 211/00

(52) U.S. Cl. 524/99; 546/184

(57) ABSTRACT

This invention relates to salt reaction products resp. premanufactured mixtures of compounds, which contain one or more groups of formula (I), and organic acids resp. their derivatives, which contain one or more groups of formula (II), wherin the symbols have the meanings as given in claim I of the application. The invention further relates to a process for the preparation of saltlike reaction products and premanufactured mixtures. The saltlike reaction products and premanufactured mixtures can be used for stabilisation of polymers, in particularly polar polymers such as polyamides and polyesters, and for the improvement of the processing properties, heat stability, colour, gloss, surface and mechanical properties of these polymers.

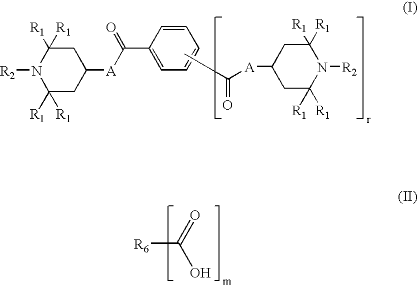

The present invention relates to new saltlike reaction products respectively premanufactured mixtures of compounds selected from the group of sterically hindered amine light stabilisers (HALS compounds) with carboxylic acids or carboxylic acid derivatives (organic acids and derivatives are in the following together called “organic acids”).

The saltlike products respectively mixtures are suitable for the heat and/or light stabilisation of polymer materials and are also suitable for the improvement of the processing and long term stability of polymers.

As known to the person skilled in the art, the stabilizing effect of HALS-compounds during exposition to mineral acids, for example originating from acid rain or from decomposition products of biocides or flame retardants, is dramatically reduced. This is considered to be mainly due to the formation of saltlike products of the HALS-compounds with mineral acids.

Surprisingly, it has now been found, that the products of the invention, although they have a saltlike character, are suitable for the stabilisation of polymers, in particular for the stabilisation of polar polymers, such as polyamide or polyester.

Moreover, the products of the invention provide for an improvement of the processing properties, such as flowability during extrusion, injection molding or fiber spinning, for an improvement of colour, gloss, surface quality and/or the mechanical properties of the final product.

The HALS-compounds are selected from the following general formula (I): 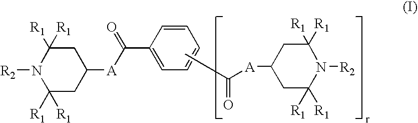
wherein independently of each other
A is —O— or —NR′—,
R′ is H, C₁-C₁₈-alkyl,
R₁ is C₁-C₁₈-alkyl, or two groups R₁ bound to the same carbon atom form a C₄-C₈-cycloalkyl ring,
R₂ is H or C₁-C₁₈-alkyl, C₇-C₁₈-alkylaryl,
r is a whole number between 0 and 3, preferably r is 1 or 2, most preferably 1;
and wherein the two carbonyl groups on the central phenyl ring are in ortho, meta or para position, preferably meta or para, most preferably meta.

The organic acids mentioned above are characterised by the general formula (I): 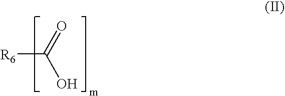
in which
independently of each other
R₆ is H, or a m-valent aliphatic, cycloaliphatic, aromatic or heteroaromatic residue,
m is a whole number>0, preferably m is 1, 2, 3 or 4; more preferably 1 or 2.

The organic acid derivatives mentioned above are those derived from formula (II), including anhydrides, cyclic esters (lactones) and particularly cyclic amides (lactames), as for example e-caprolactam.

The latter carboxylic acid derivatives after hydrolysis respectively ring opening reaction behave like mono-carboxylic acids in aqueous reaction media.

The preferred groups R₁ are methyl. Also preferred are two groups R₁ bound to the same carbon atom forming a C₄-C₆-cycloalkyl ring.

Preferred groups R₂ are H, C₁-C₁₂-alkyl or benzyl.

Preferred groups R₆ are C₁-C₁₀₀-alkyl, C₁-C₆₀-alkylene, C₄-C₁₈-cycloalkyl, C₅-C₂₄-cycloalkylene, C₆-C₁₈-aryl, C₆-C₁₈-arylene, C₅-C₁₈-heteroaryl, C₅-C₁₈-heteroarylene, C₆-C₃₆-arylalkyl, C₆-C₃₆-arylalkylene, C₆-C₃₆-alkylaryl,C₆-C₃₆-alkylarylene;

A more preferred embodiment of the invention is a saltlike compound with the general formula (HID 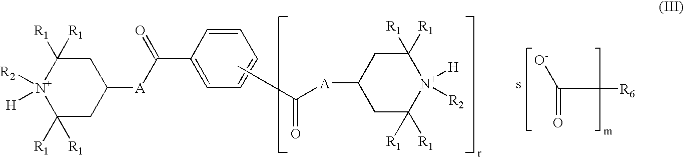
in which
r is a whole number between 0 and 3,
s represents (r+1)/m and
m is as described above,

Formula (IV) represents a particularly preferred candidate of the compound (III) 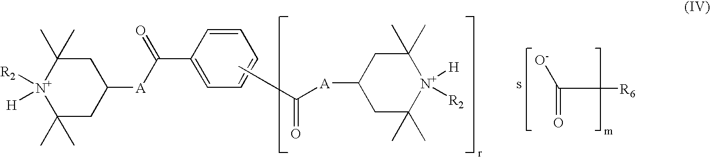
wherein the residues are the same as described above.

Formula (V) represents the most preferred candidate of the compound (III) 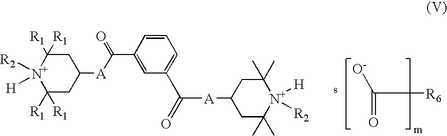
wherein r is 1 and
wherein the residues are the same as described above.

The products of the invention can be prepared in a reaction of one or more HALS-compounds (I) with one or more organic acids (II), where, depending on the molar ratio of the reaction partners and the reaction conditions, full- or hemi-amine salts of organic acids are formed. These salts show to some extent a considerably higher solubility in water or lower alcohols.

The products of the invention can be synthesised in solvents, preferrably in water or lower alcohols, or they can be synthesised in the melt. The obtained solutions can either be used directly or the products can be isolated by removal of the solvent. The resulting solids can be used in a subsequent process, optionally after further finishing.

The application of the products of the invention in polycondensation polymers like polyamide 6.6, PET or similar types, which are produced from a di-acid and a di-amino- or a diol component respectively, are particularly advantageous, as the products of the invention are easily and homogeneously dispersed in the reaction mixture during the polycondensation reaction due to their good solubility.

The HALS-compounds themselves on the other hand are less soluble due to their hydrophobic character and therefore are more difficult to be homogeneously dispersed.

Other members of the group of products of the invention are better suited for the use in polymer melt processing due to their lower polarity. It was found that the preferred properties can be influenced by the selection of the components (I) and (II) within a wide range.

A further object of the invention are premanufactured mixtures of compounds—so-called “blends” in the plastic industry—consisting of the components (I) and (II) in a selected molar ratio. The products of the invention according to formula (III) can thereby be partly or completely formed already during the making of such blends, or during incorporation into the polymer mass, respectively during incorporation into the raw materials.

The latter can be for example ε-Caprolactam (for PA6), AH-Salt-solution (for PA6.6) or precondensates from di-acids and dioles (for polyester).

As already known to the skilled person, such saltlike products respectively mixtures can also be formed by the use of HALS-compounds during the polycondensation and may disappear during continued polycondensation.

The premanufactured mixtures (blends) of the present invention are well defined with selected molar ratios. They are prepared by technical processes applicable also on large scale, which depending on the reaction conditions, already may partly form compounds of formula (III). Such blends, containing the appropriate amounts of component (I) and (II), may be added to the polycondensate. These premanufactured mixtures can be obtained from the melt (drop granulation techniques, prilling, extrusion, etc.), by compacting (roller compactors, tabletting, briquetting, press granulation, etc.), by granulation processes (spraying, fluidized bed granulation, drum granulation, etc.) or just by simple mixing.

The HALS-compounds, as well as the organic acids, can be mono- or oligofunctional. Preferred are HALS-compounds with one HALS-group and difunctional organic acids, HALS compounds with two or more HALS-groups and difunctional organic acids or HALS-compounds with two or more HALS-groups and monofunctional organic acids.

Depending on the requirements, the molar ratio of the HALS-groups and the organic acids groups can be between 1:99 and 99:1, preferably between 40:60 and 60:40, and more preferably between 45:55 and 55:45 and most preferably the ratio is equimolar (50:50).

Preferred carboxylic acids of formula (II) are C₁-C₈₀alkyl carboxylic acids, C₁-C₂₄ alkylene dicarboxylic acids, C₅-C₁₈cycloalkyl carboxylic acids, C₅-C₁₈cycloalkylene dicarboxylic acids, C₆-C₁₈aryl carboxylic acids, C₆-C₂₀arylene dicarboxylic acids, C₂-C₁₂alkyl carboxylic anhydrides, C₇₋₁₈aryl carboxylic anhydrides or C₆-C₁₈lactames.

Particularly preferred carboxylic acids or carboxylic acid derivatives of formula (II) are C₁-C₆₀alkyl carboxylic acids, C₁-C₁₈alkylen dicarboxylic acids, C₆-C₁₂cycloalkyl carboxylic acids, C₆-C₁₂cycloalkylene dicarboxylic acids, C₆C₁₂aryl carboxylic acids, C₆-C₁₂arylene dicarboxylic acids, caprolactame, laurinlactame, C₁-C₆alkyl carboxylic anhydrides or C₆-C₁₈aryl carboxylic anhydrides.

The most preferred HALS-compound of formula (I) is 1,3-Benzoldicarboxamid-N,N′-bis(2,2,6,6-tetramethyl-4-piperidinyl) (which is commercially available from Clariant as Nylostab® S-EED®).

Especially preferred carboxylic acids of formula (II) are adipic acid, terephthalic acid, isophthalic acid, phthalic acid, benzoic acid and acetic acid.

Preferred polymers for the application of the products of the invention are polyamides, like polyamide 6, polyamide 11, polyamide 12, polyamide 4.6, polyamide 6.6, polyamide 6.9, polyamide 6.10, polyamide 6.12, polyamide 12.12, and polyester like terephthalic acid/alkyldiol based polyester (for example PET, PBT), aliphatic polyester, aromatic polyester, liquid crystal polyester, polyetherester, and polycarbonates as well as their blends and copolymers.

Especially preferred polymers are polyamide 4.6, polyamide 6.6, polyamide 6, polyamide 11, Polyamide 12, polyethylenterepthalate (PET), polybutylen-terephthalate (PBT), polycarbonate, their copolymers and blends.

The addition of the products of the invention can either take place before or during the preparation of the polymer as well as during further processing steps. The products can be used as solids (powder or blend), as solutions (in inert or reactive solvents), as masterbatch or as a concentrate and, in case of un-decomposed melting compounds, also as melt.

Of particular advantage is the addition in a free flowing preparation form, as obtained for example from the melt (drop granulation techniques, prilling, extrusion, etc.) by compacting (roller compactors, tabletting, briquetting, press granulation, etc.) or by granulation processes (spraying, fluidized bed granulation, drum granulation, etc.). These manufactured forms also comprise mixtures of the products of the invention or mixtures with other polymer additives or colorants known in the art (blends).

Apart of the products of the invention, other additives or colorants can be added to the polymer before, simultaneously or afterwards, including also blends containing the products of the invention.

Examples of such other additives are lightstabilizers (further HALS-compounds, UV-absorbers, excited state quenchers), phosphor- or sulphur based processing stabilizers, antioxidants (phenol- or amine based), antistatics, nucleating agents, clarifiers, flame retardants, reinforcing materials (e.g. mineral fillers, glass fibers, hollow glass spheres, carbon fibers, nanoscale reinforcing materials (e.g. nanoclays, carbon nanotubes), lubricants, anti block agents, colorants (pigments and colorants) etc.

The production and application of the products of the invention are shown in the following examples:

### EXAMPLE1

### Synthesis of the Salt from Nylostab® S-EED® and Adipic Acid

24.8 g of adipic acid are added to a suspension of 75.3 Nylostab® S-EED® in 250 ml water and stirred at 80° C. until formation of a clear solution occurs. The water is removed under vacuum and the colorless, crystalline product is dried at 80° C. in vacuum.

### EXAMPLE 2

### Synthesis of the Salt from Nylostab® S-EED® and Terephthalic Acid

Same as EXAMPLE 1 with 72,7 g Nylostab® S-EED® and 27.3 g terephthalic acid.

### EXAMPLE 3

### Synthesis of the Salt from Nylostab® S-EED® and Benzoic Acid

Same as EXAMPLE 1 with 64.2 g Nylostab® S-EED® and 35.4 g benzoic acid.

### EXAMPLE 4

### Synthesis of the Salt from Nylostab® S-BED® and Acetic Acid

Same as EXAMPLE 1 with 78.4 g Nylostab® S-EED® and 21.2 g acetic acid.

### EXAMPLE 5

A powder mixture of 30% Nylostab S-EED and 70% terephthalic acid is transformed into a stabile, colourless and granulate on a Bepex roller compactor

### EXAMPLE 6

A powder mixture of 30% Nylostab S-EED and 70% adipic acid is transformed into a stabile, colourless and free flowing granulate on a Bepex roller compactor

### EXAMPLE 7-10

### Application of the Products of the Invention in Polyamide

General Processing Recipe

The products of the invention are homogenised together with the polyamide on a Collin single screw extruder at the mentioned temperature of use. The current of the extruder motor (equivalent to torque), the pressure at the die, the mass temperature as well as the throughput are registered at constant machine settings. The obtained polymer strand is pelletized and further used as described below.

A lower motor current and/or an increased throughput are important data, as they directly influence the production costs. A constant pressure at the die indicates good intake and feeding properties and is of importance for the reproducible preparation of end use articles, as for example, injection molding articles or especially for very uniform fibers. An increased pressure besides induces less damage of the polyamide through chain destruction during the process; the inventive additive contributes in this case to the polymer stability.

The results of the various applications obtained are summarised in the following table.

| | | | | | average | | |
|---|---|---|---|---|---|---|---|
| | | | | | motor | | Through |
| | | | | Process | current | Pressure | put |
| Example | Compound | Conc. | Polymer | Temperature | [A] | [bar] | [kg/h] |
| 7 | None | 0% | PA 6 | 250 | 4.7 ± 0.2 | 50.8 ± 1.4 | 5.45 |
| 8 | EXAMPLE 1 | 0.2% | PA 6 | 250 | 4.5 ± 0.3 | 51.9 ± 1.2 | 6.00 |
| 9 | None | 0% | PA 6.6 | 270 | 5.3 ± 2.3 | 16.3 ± 7.0 | 4.16 |
| 10 | EXAMPLE 1 | 0.2% | PA 6.6 | 270 | 3.8 ± 0.6 | 21.7 ± 1.2 | 5.00 |

### EXAMPLES 11-18

Weatherfastness and heatstability of polyamides containing the products of the invention according to EXAMPLE 1:

General Processing Recipe

The polymer granulates described above in examples 7-10 are processed into 100×100×1 mm³ plaques on an Arburg Allrounder Injection Molding Machine. The parameters of the machine are kept constant for every polymer types, respectively. The obtained plaques are tested to show the light—and heat stabilisation by the invented compound of EXAMPLE 1.

### EXAMPLE 11-14

Colour modification after exposition in Weather-O-meter according to ISO 11341 A (wet-/dry cycles) resp. ISO 11341 C (dry conditions)

| | | | | ΔE* after | ΔE* after |
|---|---|---|---|---|---|
| | | | | 2500 h | 2500 h |
| | | | | (ISO | (ISO |
| Example | Compound | Conc. | Polymer | 11341 A) | 11341 C) |
| 11 | None | 0% | PA 6 | 25.6 | 8.2 |
| 12 | EXAMPLE 1 | 0.2% | PA 6 | 10.4 | 1.6 |
| 13 | None | 0% | PA 6.6 | 12.86 | 4.4 |
| 14 | EXAMPLE 1 | 0.2% | PA 6.6 | 2.64 | 2.4 |

### EXAMPLE 15-18

Yellowing after oven ageing at 120° C.

| | | | | b* after 15 days |
|---|---|---|---|---|
| Example | Compound | Conc. | Polymer | at 120° C. |
| 15 | None | 0% | PA 6 | 23.4 |
| 16 | EXAMPLE 1 | 0.2% | PA 6 | 18.2 |
| 17 | None | 0% | PA 6.6 | 26.2 |
| 18 | EXAMPLE 1 | 0.2% | PA 6.6 | 23.1 |

1. Saltlike reaction product formed by reacting one or more components of formula (I), 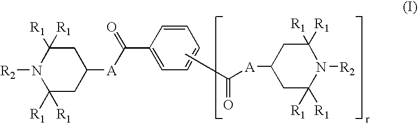
wherein independently of each other
A is —O— or —NR′—,
R′ is H, or C₁-C₁₈-alkyl,
R₁ is C₁-C₁₈-alkyl, or two groups R₁ bound to the same carbon atom form a C₄-C₈-cycloalkyl ring,
R₂ is H C₁-C₁₈-alkyl, or C₇-C₁₈-alkylaryl,
r is a whole number between 0 and 3.
and
one or more organic acids of the formula (II), 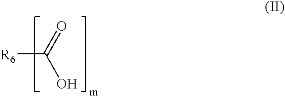
wherein independently of one another
R₆ is H, or a m-valent aliphatic, cycloaliphatic, aromatic or heteroaromatic residue,
m is a whole number >0.

2. Saltlike reaction product according to claim 1 of the general formula (III) 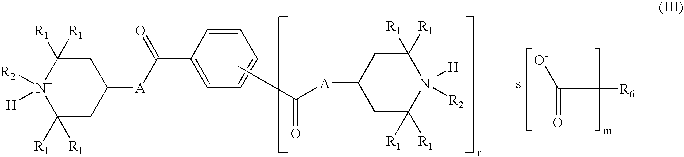
wherein
A, R₁, R₂and R₆are as defined as in claim 1,
r is a whole number between 0 and 3,
s represents (r+1)/m and
m is a whole number >0

3. Saltlike reaction product according to claim 1 of the general formula (IV) 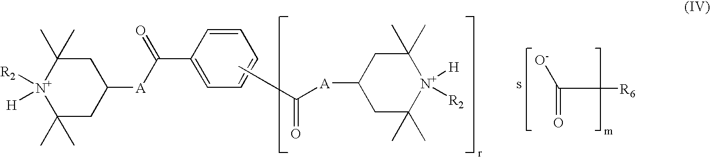
wherein
r is a whole number between 0 and 3,
s represents (r+1)/m and
A, R₂, R₆ and m are defined as in claim 1.

4. Saltlike reaction product according to claim 1 of the general formula (V) 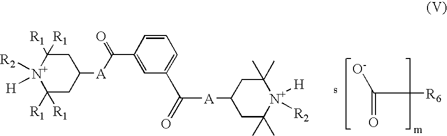
wherein
r is 1 and
s represents (r+1)/m and
A, R₂, R₆ and m are defined as in claim 1.

5. Saltlike reaction product according to claim 1 wherein the molar ratio of the one or more components of formula (I) to the one or more organic acids is 1:99 to 99:1.

6. Saltlike reaction product according to claim 1, wherein the at least one component of formula (I) is 1,3-benzoldicarboxamid-N,N′-bis(2,2,6,6-tetramethyl-4-piperidinyl).

7. Saltlike reaction product according to claim 1, wherein the at least one organic acid of formula (II) is selected from the group consisting of adipic acid, terephthalic acid, isophthalic acid, phthalic acid, benzoic acid and acetic acid.

8. Process for the preparation of a saltlike reaction products comprising the step of reacting one or more HALS-compounds of general formula (I) 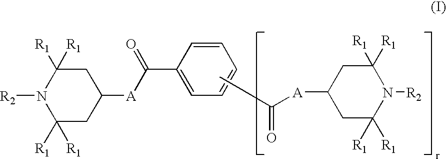
wherein independently of each other
A is —O— or —NR′—,
R′ is H, or C₁-C₁₈-alkyl,
R₁ is C₁-C₁₈-alkyl, or two groups R₁ bound to the same carbon atom form a C₄-C₈-cycloalkyl ring.
R₂ is H C₁-C₁₈-alkyl, or C₇-C₁₈-alkylaryl.
r is a whole number between 0 and 3.
with one or more organic acids of general formula (II) 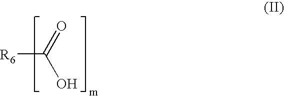
wherein independently of one another
R₆ is H, or a m-valent aliphatic, cycloaliphatic, aromatic or heteroaromatic residue,
m is a whole number >0.

9. Process for the preparation a premanufactured mixture, wherein the mixture is formed by the reaction one or more HALS-compounds of general formula (I) 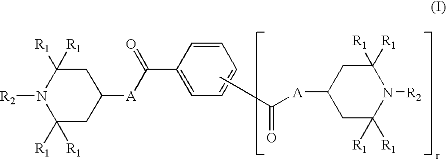
wherein independently of each other
A is —O— or —NR′—.
R′ is H, or C₁-C₁₈-alkyl.
R₁is C₁-C₁₈alkyl, or two groups R₁ bound to the same carbon atom form a C₄-C₈-cycloalkyl ring.
R₂ is H C₁-C₁₈-alkyl, or C₇-C₁₈-alkylaryl,
r is a whole number between 0 and 3
and one or more organic acids of general formula (II) 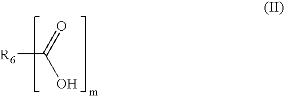
wherein independently of one another
R₆ is H, or a m-valent aliphatic, cycloaliphatic, aromatic or heteroaromatic residue,
m is a whole number >0
comprising the step of preparing the mixture, wherein the preparing step includes one of obtaining the mixture from a melt, compacting the mixture, granulating the mixture or mixing the mixture.

10. A polymer stabilizer comprising at least one reaction product according to claim 1.

11. Saltlike reaction product according to claim 1, wherein the molar ratio of the one or more components of formula (I) to the one or more organic acids is 40:60 to 60:40.

12. Saltlike reaction product according to claim 1, wherein the molar ratio of the one or more components of formula (I) to the one or more organic acids is 45:55 to 55:45.

13. Saltlike reaction product according to claim 1, wherein the molar ratio of the one or more components of formula (I) to the one or more organic acids is 50:50.

14. A saltlike reaction product made by the process of claim 8.

15. The polymer stabilizer according to claim 10, wherein the polymer to be stabilized is a polar polymer.

16. The polymer stabilizer according to claim 10, wherein the polymer to be stabilized is a polyester or a polyamide.

17. The polymer stabilizer according to claim 10, wherein the polymer to be stabilized is a polyamide.
